(19) United States
(12) Patent Application Publication
(10) Pub. No.: US 2008/0280353 A1
(43) Pub. Date: Nov. 13, 2008

(54) Hybrid nucleic acid assembly

(75) Inventors: Michael L. Weiner, Webster, NY (US); Patrick R. Connelly, Rochester, NY (US); Andrew W. Custer, Davis, CA (US)

Correspondence Address: BUCHANAN, INGERSOLL & ROONEY PC, POST OFFICE BOX 1404, ALEXANDRIA, VA, 22313-1404, US

(73) Assignee: Biomed Solutions LLC, New York, NY (US)

(21) Appl. No.: 11/902,503

(22) Filed: Sep. 21, 2007

Related U.S. Application Data
(63) Continuation of application No. 10/758,875, filed on Jan. 16, 2004, now abandoned.
Application No. 10/758,875 is a continuation-in-part of application No. 09/958,652, filed on Oct. 10, 2001, now abandoned.
(60) Provisional application No. 60/184,120, filed on Feb. 18, 2000.

(51) Int. Cl. C12M 1/00

(52) U.S. Cl. 435/287.2

(57) ABSTRACT

A detection device comprised of a hybrid nucleic acid assembly. The device further comprises a nucleic acid polymer, a first nanoparticle conjugated to the nucleic acid polymer, a second nanoparticle conjugated with the nucleic acid polymer, a means for introducing energy into the first nanoparticle, means for detecting energy from the second nanoparticle, and means for determining a physical property of the nucleic acid polymer, wherein the means of introducing energy into the first nanoparticle is a source of mechanical energy.

### CROSS-REFERENCE TO RELATED PATENT APPLICATIONS

This application is a continuation of application Ser. No. 10/758,875 filed Jan. 16, 2004, the contents of which are incorporated by reference herein, which is a continuation-in-part of application Ser. No. 09/958,652 filed Oct. 10, 2001, which is a National Stage filing under §371 of International Application No. PCT/US01/05139 filed Feb. 17, 2001, which claims the benefit of U.S. provisional Application No. 60/184,120 filed Feb. 18, 2000.

### FIELD OF THE INVENTION

The invention relates in one embodiment to an assembly comprising a strand of nucleic acids joined to a signal transceiver, and more particularly to such an assembly comprising a an electrically conductive carbon nanotube device.

An assembly comprising a strand of nucleic acids joined to a signal transceiver, such as an electrically conductive carbon nanotube device, is provided.

### BACKGROUND OF THE INVENTION

In an article by Hans-Werner Fink and Christian Schonenberger, published in Nature (Volume 398, Apr. 1, 1999, at pages 407-410), the authors stated that: “The question of whether DNA is able to transport electrons has attracted much interest . . . . Experiments addressing DNA conductivity have involved a large number of DNA strands doped with intercalated donor and acceptor molecules, and the conductivity has been assessed from electron transfer rates as a function of the distance between the donor and acceptor sites. But the experimental results remain contradictory, as do theoretical predictions.”

The prior art techniques for measuring DNA conductivity are relatively crude, with many of such techniques, the acts of measurement influence the very variables being measured. Additionally, to the best of applicants' knowledge, none of the prior studies of DNA conductivity measured such conductivity with the DNA in an environment similar to its natural environment.

It is an object of this invention to provide a process for measuring DNA conductivity which is substantially more accurate than prior art processes.

It is another object of this invention to provide a process for measuring DNA conductivity (of electrons, photons, and vibration) while such DNA is undergoing its normal processes (such as transcription or replication) in substantially its normal environment.

It is another object of this invention to provide a process for measuring the shape structure of DNA.

It is another object of this invention to provide a novel hybrid nucleic acid assembly useful in practicing the processes of this invention.

### SUMMARY OF THE INVENTION

In accordance with the present invention, there is provided detection device comprised of a hybrid nucleic acid assembly, wherein said hybrid nucleic acid assembly is comprised of a nucleic acid polymer, a first nanoparticle conjugated to said nucleic acid polymer, a second nanoparticle conjugated to said nucleic acid polymer, a means for introducing energy into said first nanoparticle, means for detecting energy from said second nanoparticle, and means for determining a physical property of said nucleic acid polymer.

In accordance with the present invention, there is further provided a detection device comprised of a hybrid amino acid assembly, wherein said hybrid amino acid assembly is comprised of an amino acid polymer comprised of a proximal end and a distal end, a first antibody binding to a first antigenic site on said amino acid polymer, a second antibody binding to a second antigenic site on said amino acid polymer, a first nanoparticle conjugated with said first antibody, a second nanoparticle conjugated with said second antibody, means for introducing energy into said first nanoparticle, means for withdrawing energy from said second nanoparticle, means for detecting the withdrawal of energy from said second nanoparticle, and means for determining a physical property of said amino acid polymer while said energy is introduced into said first nanoparticle.

In accordance with the present invention, there is further provided a dendrimer-nucleic acid-energy detector complex wherein said complex is comprised of a dendrimer, a nucleic acid sequence and an energy detection device.

In accordance with the present invention, there is further provided a dendrimer-polypeptide-energy detector complex wherein said complex is comprised of a dendrimer, a polypeptide sequence and an energy detection device.

### BRIEF DESCRIPTION OF THE DRAWINGS

The invention will be described by reference to the following drawings, in which like numerals refer to like elements, and in which:

FIG. 1A is a flow diagram of one preferred process of the invention;

FIG. 1B is a flow diagram of a process for making certain transceivers;

FIGS. 2A, 2B, and 2C are schematic representations of various transceiver assemblies which may be used in the process of the invention;

FIGS. 3A and 3B are schematic diagrams of a measurement device utilizing the hybrid nucleic acid assembly of FIGS. 2A-2C;

FIG. 4A is a schematic diagram of a further embodiment similar to the measurement device of FIG. 3A, wherein the device is connected to the hybrid nucleic acid assembly at tubulin protein subunits thereof;

FIG. 4B is a schematic diagram of a further embodiment similar to the measurement devices of FIG. 3B and FIG. 4A, wherein fluorophores are attached to the tubulin subunits within a cell;

FIG. 5A is a schematic diagram of a further embodiment similar to the measurement device of FIG. 3A, wherein both electrode ends of the device are connected to antibodies or antibody fragments that recognize a given antigen or antigens;

FIG. 5B is a schematic diagram of a further embodiment similar to the measurement device of FIG. 3A, wherein both electrode ends of the device are connected to a single protein;

FIG. 5C is a schematic diagram of a further embodiment similar to the measurement device of FIGS. 4B and 5B, wherein fluorophores are attached to the single protein;

FIG. 6 is a flow diagram of an imaging process for detecting the shape structure of DNA;

FIG. 7A is an example of star dendrimer structure conjugated to a specific nucleic acid sequences;

FIG. 7B is a partial view of two star dendrimers conjugated to a cDNA; and

FIG. 8 is an example of starburst dendrimer structures linked to the microtubule network in the cytoskeleton of a representative cell.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by the appended claims.

### DESCRIPTION OF THE PREFERRED EMBODIMENTS

For a general understanding of the present invention, reference is made to the drawings. In the drawings, like reference numerals have been used throughout to designate identical elements. In describing the present invention, a variety of terms are used in the description.

As used herein, the term oligonucleotide is meant to indicate a molecule usually composed of (but not limited to) 25 or fewer nucleotides, such as is sometimes used as a DNA synthesis primer.

As used herein, the term “oligonucleotide activation”, as used in this specification, refers to chemical modification of the nucleic acid(s) within the oligonucleotide to enable such nucleic acid(s) to react with another molecule located on a transceiver. One may activate the oligonucleotide by conventional means. Thus, e.g., one may use alkylthio functionalization activation of DNA; see, e.g., an article by Gregory P. Mitchell et al. Entitled “Programmed Assembly of DNA Functionalized Quantum Dots” (Journal of the American Chemical Society, 1999, 121, pages 81232-8123). Thus, one may use methylation of DNA. Thus, e.g., one may use the techniques described in an article by Jens-Peter Knemeyer et al. Entitled “Probes for Detection of Specific DNA Sequences . . . ,” appearing in Analytical Chemistry, Volume 72, pages 3717 to 3724 (Aug. 15, 2000). Thus, e.g., one may use one or more of the activation techniques described in U.S. Pat. Nos. 6,013,789, 5,577,694, 5,747,244, 5,712,383, 5,612,468, 5,525,711, and the like; the entire disclosure of each of these United States patents is hereby incorporated by reference into this specification.

As used herein, the term “reactive surface” refers to any surface which is capable of binding activated oligonucleotides. Such a surface may be any surface commonly used in the preparation of DNA chips, such as derivatized glass, silicon, or any polymeric surface. The preparation of derivatized reactive surfaces is well known; see, e.g., U.S. Pat. Nos. 5,641,539, 5,453,199, 5,372,719, and the like, the entire disclosures of each of which is hereby incorporated by reference into this specification. In addition, such surfaces are routinely prepared specifically for the attachment of activated oligonucleotides, for example in U.S. Pat. Nos. 6,413,722 and 6,399,365, the entire disclosures of each of which are hereby incorporated by reference into this specification.

As used herein, the term “transceiver” refers to any molecule, compound, structure, or article capable of either conducting a signal, transducing a signal from one form to another, initiating a signal, etc.

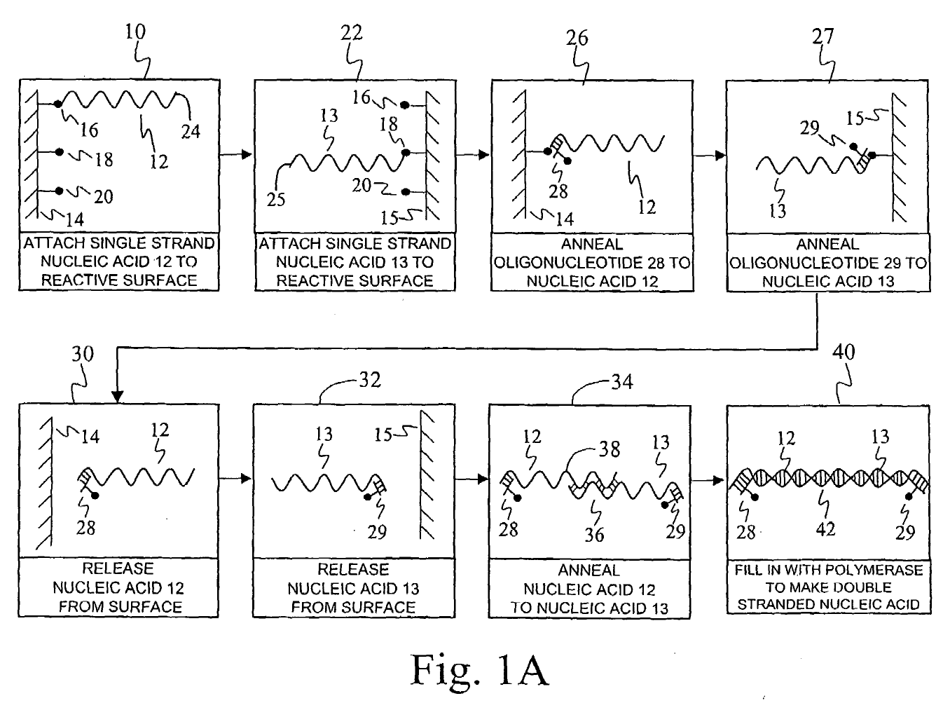

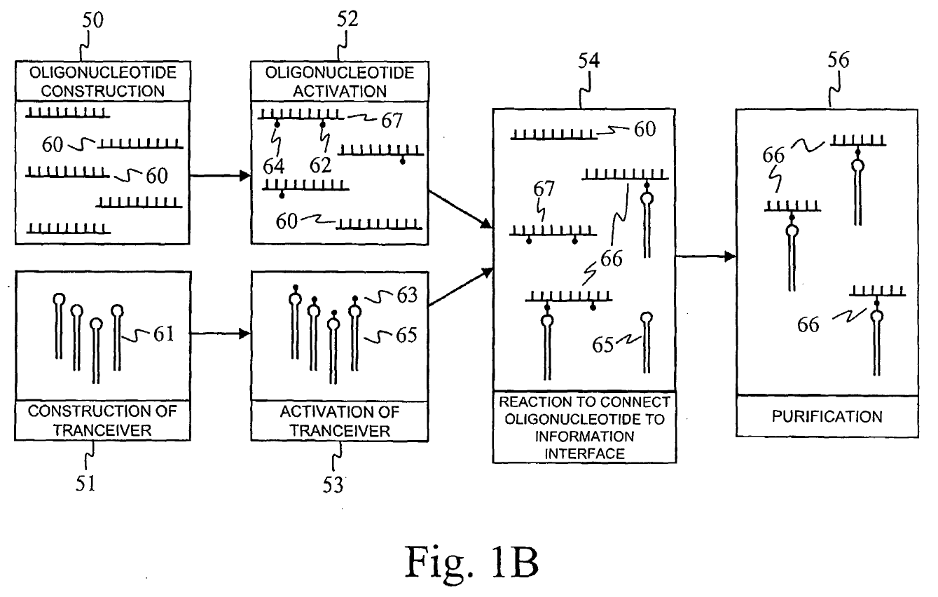

FIG. 1A is a flow diagram of one preferred process of the invention. FIG. 1B is a schematic diagram illustrating how preferred oligonucleotide assemblies used in the process of FIG. 1A may be assembled.

Referring to FIG. 1A, and in the preferred embodiment depicted therein, in step 10 of the process, a single strand of nucleic acid 12 is attached to a reactive surface 14 comprised of reactive sites 16, 18, and 20.

One may prepare single stranded DNA by conventional means, such as, e.g., complementary DNA (cDNA) preparation techniques; see, e.g., U.S. Pat. Nos. 6,184,017, 6,180,612, 6,180,385, 6,177,244, and 6,172,197, the entire disclosure of each of which is hereby incorporated by reference into this specification.

The single stranded DNA, 12, used in the process depicted in FIG. 1A is the sequence to be analyzed. It may, e.g., contain promoter and/or enhancer regions, structural genes (including introns, exons, non-coding DNA, “Junk DNA” and the like), etc.

Referring again to FIG. 1A, it is preferred that the single stranded DNA 12 be purified, i.e., it be substantially homogeneous. DNA purification may be effected by conventional means; see, e.g., U.S. Pat. Nos. 6,187,578, 6,187,575, 6,187,564, 6,187,559, 6,187,552, and the like, the entire disclosure of each of which is hereby incorporated by reference into this specification.

Reactive surface 14, as depicted in FIG. 1A, may be any surface commonly used in the preparation of DNA chips. Such DNA chips are well known and are sold, e.g., by the Affymetrix Company, by the Incyte Company, etc. Alternatively, the reactive surface 14 may be a passive derivatized polymeric or glass surface. The preparation of derivatized reactive surfaces is well known; see, e.g., U.S. Pat. Nos. 5,641,539, 5,453,199, 5,372,719, and the like, the entire disclosures of each of which is hereby incorporated by reference into this specification.

In one embodiment and as depicted in FIG. 1A, the reactive surface 14 is described in an article by Brett A. Stilman et al. Entitled “FAST Slides: A Novel Surface for Microarrays” appearing in the September, 2000 edition (Volume 29, No. 3) of BioTechniques, at pages 630-635.

Referring again to FIG. 1A, in step 10, one may attach single stranded DNA 12 to reactive surface 14 by conventional techniques. Thus, by way of illustration and not limitation, one may attach strand 12 to surface 14 by covalent bonding. See, e.g., U.S. Pat. Nos. 5,472,888 and 6,177,247, the entire disclosure of each of which is hereby incorporated by reference into this specification.

In step 22, another strand of preferably purified DNA 13 is attached to reactive surface 15, which preferably has characteristics similar to surface 14 but may differ therefrom. It is preferred that DNA strand 13 have a base sequence that differs from DNA strand 12. As will be apparent to those skilled in the art, DNA strand 13 also is derived from the DNA to be analyzed.

In another embodiment (not shown), two different reactive surfaces are used to bond to DNA strands 12 and 13, respectively.

In one embodiment, strands 12 and 13 have similar base sequences. What is required however, in all embodiments, is that end 24 of strand and end 25 of strand 13 have complementary base pairs to facilitate annealing therebetween.

In one preferred embodiment, and as depicted in FIG. 1A, each of steps 10 and 22 occur in different environments, such as, e.g., separate test tubes. In this embodiment, two distinct, noncontiguous reactive surfaces are used.

In step 26 of the process, an oligonucleotide 28 is annealed to DNA strand 12 by conventional means; see, e.g., U.S. Pat. Nos. 6,083,723, 6,083,698, 6,051,379, 6,017,731, 5,972,604, and the like, the entire disclosure of each of which is hereby incorporated by reference into this specification. As will discussed in detail elsewhere in this specification, oligonucleotide 28 (and oligonucleotide 29 in step 27) is attached to a device, which is capable of generating an electrical or magnetic or optical signal or otherwise transmitting information.

It is preferred that oligonucleotide 28 contains base pairs complementary to the base pairs in the region of DNA strand 12 to which oligonucleotide 28 is to be annealed. Similarly, oligonucleotide 29 preferably contains base pairs complementary to a region of DNA strand 13 and is annealed to DNA strand 13 in step 27.

In steps 30 and 32, the annealed DNA strands 12 and 13 are optionally caused to be released from reactive surfaces 14 and 15, respectively. This is preferably done by the breaking of the chemical bond between strands 12 and 13 and surfaces 14 and 15, respectively. This breaking may be effected by conventional means such as, e.g., restriction enzyme cleavage.

In one embodiment, step 30 and/or step 32 is omitted. In one embodiment, only one of strands 12 or 13 is released. Thus, e.g., one may have a situation in which the bond energy between strand 12 and surface 14 is substantially greater than the bond energy between strand 13 and surface 15, in which case the latter bond is preferentially broken after strands 12 and 13 are hybridized. By way of illustration, surfaces 14 and 15 may exist on a flat or spherical surface (such as, e.g., beads).

One may cleave the DNA strands 12 and 13 by conventional means such as, e.g., those disclosed in U.S. Pat. Nos. 6,183,993, 6,180,402, 6,180,338, 6,175,001, 6,174,724, and the like; the entire disclosure of each of these United States patents is hereby incorporated by reference into this specification.

The released strands 12 and 13 that are bonded to oligonucleotides 28 and 29, respectively, are then preferably pooled by charging them to the same container, which preferably contains a buffer, such as tris-buffer. Thereafter, in step 34, the released strands 12 and 13 anneal to each other in mutually complementary region.

The annealed structure 38 is partially single stranded and partially double stranded. In step 40, annealed structure 38 is made completely double stranded by exposing it to base building blocks (nucleotides) in the presence of polymerase and ligase, in accordance with standard protocols for DNA vector construction. See, e.g., U.S. Pat. Nos. 4,853,323, 6,184,034, 6,184,000, 6,177,543, 6,171,861, and the like; the entire disclosure of each of these United States patents is hereby incorporated by reference into this specification.

In the embodiments depicted in FIG. 1A, oligonucleotides are depicted as being disposed on opposite strands 12 and 13. In another embodiment, not shown, the oligonucleotides are disposed on the same strands. In yet another embodiment, not shown, the oligonucleotides are disposed on both the same and on opposite strands. Other combinations will be apparent to those skilled in the art.

Referring to step 40 of FIG. 1A, the double-stranded structure 42 produced by this process has incorporated within it oligonucleotides 28 and 29.

FIG. 1B illustrates one preferred process for constructing oligonucleotide devices (e.g. oligonucleotides 28 and 29 in FIG. 1A) and, attached to each such entity, an information interface. In step 50 of this process, the desired oligonucleotide 60 with the desired base sequence(s) is constructed by conventional means and/or purchased.

In step 52 of the process depicted in FIG. 1B, the oligonucleotide is activated at sites 62 and 64 to produce activated oligonucleotide 67; as will be apparent to those skilled in the art, the oligonucleotide activation step is variable. Some of the oligonucleotides are not activated, some are activated at one site, and some are activated at two or more sites.

A suitable transceiver 61, as defined below, is constructed in step 51, and the transceiver 61 is activated in step 53 at site 63 to become activated transceiver 65, i.e. the surface of transceiver 61 is activated in step 53.

By way of illustration, the transceiver may be a metal fiber such as, e.g., a gold fiber. By way of further illustration, the transceiver may be a carbon nanotube fiber, an electrically conductive polymer, a chromophor (which changes its optical properties upon excitation), a fluorophor (which also changes its optical properties upon excitation), a lumiphor (which emits photons upon receipt of electrons), a molecular battery (which releases electricity upon photon stimulation), a radio frequency antenna (which receives or transmits radio frequency energy upon excitation), and the like. The transceiver may also transmit and receive some type of mechanical energy. The mechanical energy may be in the form of ultrasonic energy. Additionally, the transceiver may be comprised of a radioactive material whose decay initiates one or more chemical reactions, resulting in the discharge of electrons. The transceiver may be a dendrimer or nanoparticle of any composition with the properties just described.

Regardless of the structure of the transceiver, or its form or composition, it is preferred to activate such transceiver in step 53 so that it will readily bond to the activated oligonucleotide, preferably by chemical means. The bonding may occur by any known mechanism such as, e.g., by Van Der Waals forces, by ionic forces, by polar forces, by combinations thereof, and the like. In one preferred embodiment, the bonding is effected primarily by covalent bonds.

Nanoparticles or nanotubes can be functionalized to allow binding of activated oligonucleotides or proteins using a variety of methods. Nanotubes can be functionalized with imide groups by chemical means, as detailed in U.S. Pat. No. 6,331,262, the entire disclosure of which is herein incorporated by reference. Nanotubes may also be functionalized with sulfur, carboxylic acid, or metals. See, e.g. U.S. Pat. No. 6,203,814, the entire disclosure which is hereby incorporated by reference into this specification. One may also use the technique of plasma exposure in US Patent Application No. 2002/0197474, which will functionalize nanotubes with polyimide groups; the entire disclosure of which is hereby incorporated by reference into this specification.

Synthesis of oligonucleotides with modified bases is well known to those skilled in the art. Nucleic acids modified with amino or thiol groups, as well as biotin can be purchased commercially or synthesized in the lab. Oligonucleotides formed using such modified nucleic acids can then be crosslinked to amine- or thiol-functionalized nanoparticles using compounds such as N-Sulfosuccinimidyl[4-iodoacetyl]aminobenzoate (Sulfo-SIAB), which reacts to both sulfhydryl and amino groups. A variety of similar cross-linking agents may be used, as is known to one skilled in the art.

It may be desirable to extend the linkage from the nanoparticle, nanotube or dendrimer in order to avoid steric effects between the oligonucleotide or protein and the nanoparticle. U.S. Pat. No. 6,479,240 describes electrically conductive oligomers which can link an electrode and nucleic acids, the entire disclosure of which is hereby incorporated by reference into this specification.

In another embodiment, electrically-conductive dendrimers can be used in place of nanoparticles as a conductive material. U.S. Pat. No. 6,043,336 discloses dendrimers with functionally active amino terminii which may be modified with naphthalene diimide or mono-anhydride mono-imide compounds. The modified dendrimers then contact a reducing agent, causing anion radical groups to form and allowing pi-stacking in aqueous or polar solutions. U.S. Pat. No. 6,020,457 discloses dendrimers that contains sulfhydryl groups which may be reduced, allowing attachment of proteins via disulfide linkages through cysteines. Conjugation of oligonucleotides to dendrimers via a hexamethylene amine group on the 5′ end of the oligonucleotide is also disclosed.

Referring again to FIG. 1B, and in the preferred embodiment depicted therein, in step 54 the activated oligonucleotide 67 is reacted with the activated transceiver 65 to produce a hybrid assembly 66 adapted to receive information from or transmit information to a DNA sequence.

In step 56, the hybrid assembly products 66 produced in step 54 may be purified by conventional means such as, e.g., chromatography to insure that the reaction products are substantially homogeneous. See, e.g., U.S. Pat. Nos. 6,187,578, 6,187,585, 6,187,564, 6,187,559, and 6,187,552; the entire disclosure of each of these United States patents is hereby incorporated by reference into this specification.

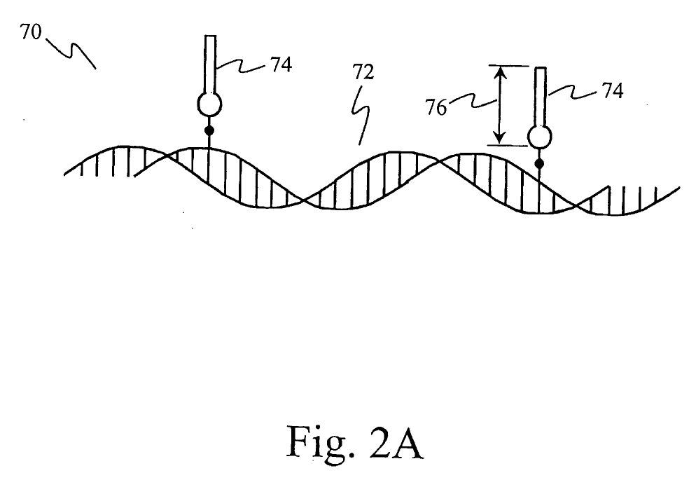

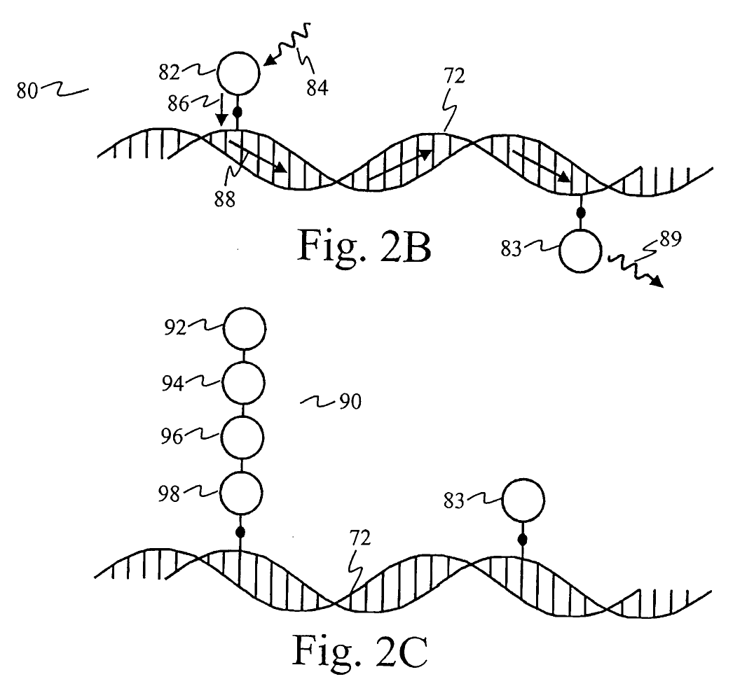

FIG. 2A is a representation of a transceiver assembly 70 comprised of a double stranded DNA 72 produced in accordance with steps 26-40 of the process depicted in FIG. 1A. In the embodiment depicted in FIG. 2A, the transceivers 74 are carbon nanotubes that have a length 76 such that they preferably extend out of the environment in which the double stranded DNA 72 is disposed. Thus, for example, if the DNA 72 is disposed within a test tube (not shown), the tops of transceivers 74 preferably extend beyond the top of the test tube. As will be apparent, this feature enables other devices to be readily attached to the tops of the nanotubes 74; and it also facilitates the use of such other devices whose use might be interfered with, given the physical constraints of the test tube.

In the embodiment 70 depicted in FIG. 2A, each of the transceivers 74 is a carbon nanotube. In the transceiver assembly 80 depicted in FIG. 2B, each of the transceivers 82 and 83 is a molecule which, preferably, is a fluorophore (a potentially fluorescent group of atoms in a molecule) which will fluoresce upon being excited; see, e.g., U.S. Pat. Nos. 6,187,567, 6,187,566, 6,187,250, 6,184,027, 6,183,984, and the like, the entire disclosure of each of which is hereby incorporated by reference into this specification. Transceivers 82 and 83 may consist essentially of the same material, or they may consist essentially of different materials.

If a photon 84 impinges upon the surface of fluorophoric transceiver 82, the transceiver 82 will emit an electron (not shown) which will travel into the double stranded DNA 72 in the direction of arrow 86 and thence through the DNA 72 in the direction of arrows 88 until it is received by receptor transceiver 83, which attracts the electron because of a potential difference therewith. When the electron is received by transceiver 83, it will emit a photon 89, fluoresce, and then return to its base state potential which existed before it received the electron. As will be apparent, this device enables the construction of more complicated circuits.

The embodiment depicted in FIG. 2C is similar to that depicted in FIG. 2B with the exception that the transceiver assembly 90 is comprised of a multiplicity of fluorophoric sites 92, 94, 96, and 98. When such a transceiver assembly 90 is excited by a multiplicity of electrons (not shown), it will emit substantially more electrons than the single fluorophore depicted in FIG. 2B; and it will thus cause the transceiver 83 to fluoresce substantially longer and/or more frequently.

As will be apparent, a photon can cause the emission of an electron in a fluorophoric device. It will be apparent, however, that this process is reversible, and that an electron can cause the emission of a photon from a fluorophoric device.

One can determine, under normal conditions for a specified double stranded DNA, how much a specified amount of excitation energy causes a current to flow, especially if measuring devices are connected to the transceivers. Consequently, one can determine when there is any aberrant condition with such DNA that would affect such current flow, and/or one can determine when normal DNA processes (such as transcription or replication) are occurring. Reference data can be generated as to the current flows normally existing during these events, and such data can be correlated with readings taken from the DNA when it is in a substantially in vivo environment.

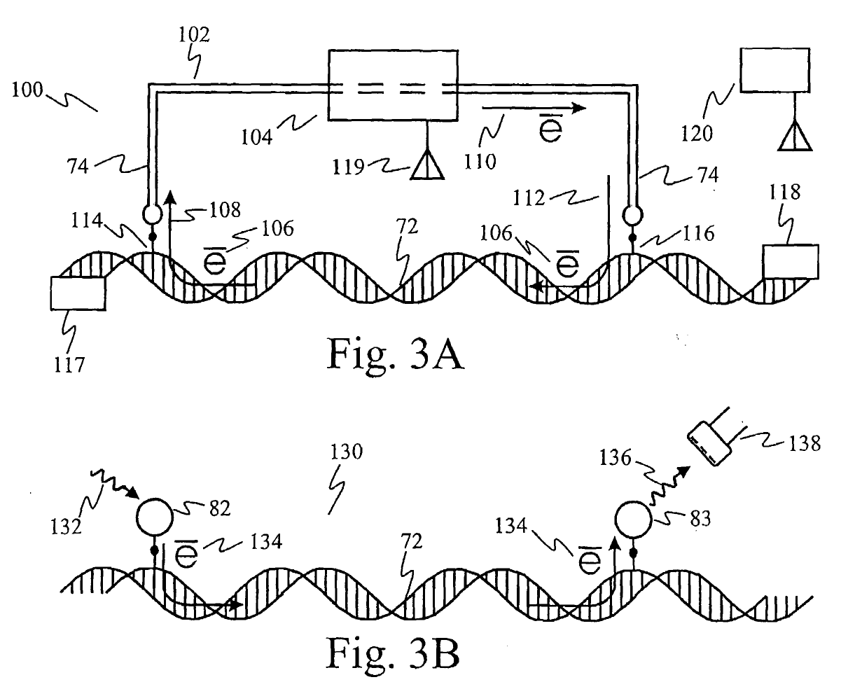

FIG. 3A is a schematic representation of a circuit 100 in which transceivers 74 are conductively connected by conductor 102 to form a closed circuit comprised of such transceivers 74, such conductor 102, and double stranded DNA 72. Disposed within such circuit 100 is controller 104 which is capable of sending energy 106 around the circuit 100 in the directions of arrows 108, 110, and 112. The energy transmitted by controller 104 may be thermal energy, and/or light energy, and/or vibrational energy, and/or magnetic energy, and/or electrical energy. Any of the forms of such energy commonly available or producible may be sent by controller 104. In a further embodiment, controller 104 is composed, in part, of a fluorophore. In another embodiment, controller 104 is composed, in part, of a quantum dot.

By way of illustration and not limitation, the energy transmitted may be electrical energy that is either direct current energy or alternating current energy. When alternating current energy is sent, it may be amplitude modulated energy, frequency modulated energy, phase modulated energy, and the like. As will be apparent, one may vary the amplitude, voltage, frequency, current, and impedance of such energy, as is well known in the electrical art.

By way of further illustration, the energy transmitted may be light energy, either in the form of waves and/or particles, at various frequencies, wavelengths, or combinations thereof. In this embodiment, the conductor 102 may be a fiber optic conductor.

The controller 104, in addition to emitting energy, also is capable of measuring the characteristics of the DNA between points 114 and 116. As will be apparent, although only two connection points 114 and 116 are depicted in FIG. 3A, more of such connections could be made so that one could determine the electrical characteristics (i.e. voltage potential) between any two points on DNA strand 72.

The electrical properties of DNA strand 72 will vary depending upon its geometry and chemical composition. These characteristics will, in turn, vary when events such as protein binding, transcription, replication, denaturation, and the like occur. Thus, the circuit 100 may be used to determine when a particular strand 72 of DNA is undergoing such an event and/or whether a particular strand of DNA 72 evidences an aberrant behavior or composition or geometry which affects such electrical characteristics. One form of aberrant behavior or composition is a missense mutation or deletion mutation within a specified gene sequence.

The controller 104 is preferably comprised of a programmable logic chip which enables it to modify its performance upon evaluation of the data it collects from DNA strand 72.

In one embodiment, the circuit 100 is disposed in an environment (not shown) which substantially simulates and/or is substantially identical to the environment the DNA strand 72 normally is in. Thus, one may charge circuit 100 to an aqueous environment comprised of buffer, essential biological components (such as nucleotides, adenosine triphosphate, protein enzymes), and the like. Thus, one may ligate circuit 100 into a DNA vector (not shown) by means of linker sequences 117 and 118, in accordance with standard biotechnical protocols; see, e.g., U.S. Pat. Nos. 6,187,757, 6,183,753, 6,180,782, 6,136,568, 6,136,318, and the like, the entire disclosure of each of which is hereby incorporated by reference into this specification. Thus, the circuit 100 may be introduced into a functional biological entity such as a bacteria or eukaryotic cell and, thereafter, used to monitor the in vivo activity within the bacterium or eukaryotic cell. As will be apparent, the vector containing circuit 100 can be made part of any nucleic entity (such as, e.g., a plasmid, a chromosome) and, after such modification, undergoes precisely the same occurrences as would an endogenous species. Thus, the circuit 100 may be used to evaluate and monitor and modify a wide variety of in vivo activities.

In another embodiment, not shown, the circuit 100 is encapsulated in a lipid derived delivery system prior to being incorporated within a cell. This technique is well known and is described, e.g., in U.S. Pat. No. 6,187,760, the entire disclosure of which is hereby incorporated by reference into this specification. As is disclosed in such patent, the introduction of foreign nucleic acids and other molecules is a valuable method for manipulating cells and has great potential both in molecular biology and in clinical medicine. Many methods have been used for insertion of endogenous nucleic acids into eukaryotic cells. E.g., see Graham and Van der Eb, Virology 52, 456 (1973) (co-precipitation of DNA with calcium phosphate); Kawai and Nishizawa, Mol. Cell. Biol. 4, 1172 (1984) (polycation and DM80); Neumann et al., EMBO Journal 1, 841 (1982) (electroporation); Graessmann and Graessmann in Microinjection and Organelle Transplantation Techniques, pp. 3-13 (Cells et al., Eds., Academic Press 1986) (microinjection); Cudd and Nicolau in Liposome Technology, pp. 207-221 (G. Gregoriadis, Ed., CRC Press 1984) (liposomes); Cepko et al., Cell 37, 1053 (1984) (retroviruses); and Schaffher, Proc. Natl. Acad. Sci. USA 77, 2163 (1980) (protoplast fusion). Both transient and stable transfection of genes has been demonstrated.

Some of the first work on liposome delivery of endogenous materials to cells occurred about twenty years ago. Foreign nucleic acids were introduced into cells (Magee et al., Biochim. Biophys. Acta 451, 610-618 (1976), Straub et al., Infect. Immun. 10, 783-792 (1974)), as were foreign lipids (Martin and MacDonald, J. Cell Biol. 70, 5 15-526 (1976)), Proteins (Magee et al., J. Cell. Biol. 63, 492 (1974), Steger and Desnick, Biochim. Biophys. Acta 464, 530 (1977)), fluorescent dyes (Leventis and Silvius), and drugs (Juliano and Stamp, Biochem. Pharm. 27, 2127 (1978), Mayhew et al., Cancer Res. 36, 4406 (1976), Kimelberg, Biochim. Biophys. Acta 448, 531 (1976)), all using positively charged lipids.

Of the many methods used to facilitate entry of DNA into eukaryotic cells, cationic liposomes are among the most efficacious and have found extensive use as DNA carriers in transfection experiments. See, generally, Thierry et al. in “Gene Regulation: Biology of Antisense RNA and DNA,” page 147 (Erickson and Izant, Eds., Raven Press, New York, 1992); Hug and Sleight, Biochim. Biophys. Acta 1097, 1 (1991); and Nicolau and Cudd, Crit. Rev. Ther. Drug Carr. Sys. 6, 239 (1989). The process of transfection using liposomes is called lipofection. Senior et al., Biochim. Biophys. Acta 1070, 173 (1991), suggested that incorporation of cationic lipids in liposomes is advantageous because it increases the amount of negatively charged molecules that can be associated with the liposome. In their study of the interaction between positively charged liposomes and blood, they concluded that harmful side effects associated with macroscopic liposomeplasma aggregation can be avoided in humans by limiting the dosage.

Feigner et al., Proc. Natl. Acad. Sci. USA 84, 7413 (1987), demonstrated that liposomes of dioleoylphosphatidylethanolamine (DOPE) and the synthetic cationic lipid N-[1-(2,3-dioleyloxy)propyl]-N,N,N-trimethylammonium chloride (DOTI VIA) are capable of both transiently and stable transfection DNA. Rose et al., BioTechniques 10, 520 (1991), tested lipofection with liposomes consisting of DOPE and one of the cationic lipids cetyldimethylethylammonium bromide (CDAB), cetyltrimethylethylammonium bromide (CTAB), dimethyldioctadecylammonium bromide (DDAB), methylbenzethonium chloride (MBC) and stearylamine. All of the liposomes (except that with CTAB) successfully transfected DNA into HeLa cells. At high concentrations; however, CDAB and MBC caused cell lysis. Only DDAB was found to be effective in mediating efficient DNA transfection into a variety of other cell lines. Malone et al., Proc. Natl. Acad. Sci. USA 86, 6077 (1989), successfully transfected RNA, in vitro, into a wide variety of cells lines. Zhou and Haung, J. Controlled Release 19, 269 (1992), disclosed successful lipofection by DOPE liposomes stabilized in the lamellar phase by cationic quaternary ammonium detergents. The authors noted, however, that the relatively high cytotoxicity of these compounds would limit their use in vivo.

Hawley-Nelson et al., Focus 15, 73 (1990, BRL publications), disclosed the cationic lipid “LIPOFECTAMINE”, a reagent containing 2,3-dioleyloxy-N-[2(spermineearboxyamido)ethyl]-N,N-dimethyl-1-propanaminium trifluoroacetate (DOSPA). “LIPOFECTAMINE” was found to have higher transfection activity than several monocationic lipid compounds (“LIPOFECTIN”, “LIPOFECTACE”, and DOTAP) in six of eight cell types tested. They observed toxicity when both lipid and DNA were included in the same mixture. These encapsulating agents are sold by Gibco BRL Systems, Inc. of Bethesda, Md.

Another possible method of introducing these constructs is to use viral vectors. U.S. Pat. No. 6,180,389 discloses the use of virion coat proteins to encapsulate and deliver organic and inorganic nanoparticles; the entire disclosure of which is hereby incorporated by reference into this specification.

Spherical dendrimers may also be used as a transfection agent. U.S. Pat. Nos. 5,714,166 and 6,475,994 disclose spherical dendrimers with a net positive charge to be used as a transfection agent. The dendrimers are prepared to contain the material to be transfected into the cell and are then introduced to the cell via particle bombardment, the so-called gene gun method. Such dendrimers may be utilized in delivering an array of differing DNA construct/circuits (100 in FIG. 3A) for increased variety in analysis. The entire disclosures of these patents is hereby incorporated by reference into this specification.

Referring again to FIG. 3A, and in the embodiment depicted therein, an antenna 119 is operatively connected to the controller 104 and is adapted to transmit signals in response to instructions from such controller. In one aspect of this embodiment, the antenna 119 emits signals in response to readings taken of the DNA strand 72. Inasmuch as the electrical properties of DNA strand 72 vary substantially instantaneously when various biochemical events occur, a remote receiver 120 disposed outside of circuit 100 may be used to monitor the status of and the activity of such DNA. This is especially useful when circuit 100 is disposed within a living being.

FIG. 3B depicts a circuit 130 similar to circuit 100 but differing therefrom in that the energy is introduced from a source remote from circuit 130. One may use any conventional remote energy source such as, e.g., a laser, sound, radio frequency energy, magnetism, and the like. In the embodiment depicted, upon the introduction of photonic energy 132 to fluorophoric transceiver 82, an electron 134 is caused to flow within the DNA strand 72 and flows to the lower potential fluorophoric transceiver 83, which it causes to emit energy in the form of a photon 136. The fluorescence may be detected by a photodetector 138 which, depending upon the intensity and frequency of the fluorescence, will monitor and measure activity within DNA strand 72.

Redox-active intercalating agents known to those familiar with the art, such as methylene blue or the intercalating agent described in US2003/0039975, may be used to improve electron movement along the double-stranded DNA. DNA modified with metals, such as M-DNA described in US2002/0175317A1 may also be used to improve conduction properties of the DNA. Peptide nucleic acids (PNA) which are capable of binding to single-stranded DNA with great specificity may also be used, as well as RNAs.

Referring to FIGS. 3A and 3B, when circuits 100 or 130 are disposed within a cell, e.g., and when the oligonucleotides 28 and 29 (refer to FIG. 1A) are disposed on opposite strands 12 and 13, when such a cell divides, each daughter cell will then receive one end of the circuit 100 or 130, in which case the circuit 100 or 130 measures conductivity across two or more cells.

Such circuits can also be connected to proteins and can measure changes in electrical properties of such proteins. Proteins and peptides can be linked to imide-functionalized nanoparticles using any one of variety of cross-linking reagents as is known to one skilled in the art, such as e.g. N-Sulfosuccinimidyl[4-iodoacetyl]aminobenzoate (Sulfo-SIAB).

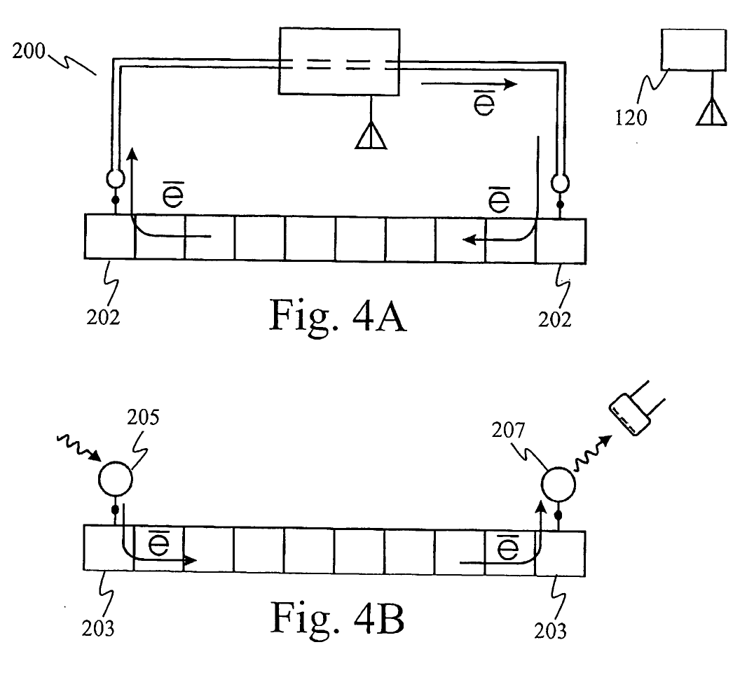

FIG. 4A is a schematic diagram of a further embodiment similar to the measurement device of FIG. 3A, wherein the device is connected to the hybrid nucleic acid assembly at tubulin subunits thereof. Referring to FIG. 4A, the device 200 is connected to tubulin subunits 202 which can then be incorporated into microtubules of a living cell. This device is similar in all ways with that depicted in FIG. 3A, except for the fact that it is connected to tubulin subunits. Such an embodiment could passively monitor possible electron flow or voltage potential along microtubules.

FIG. 4B is a schematic diagram of a further embodiment similar to the measurement device of FIG. 4A, wherein fluorophores are attached to the tubulin subunits of the hybrid nucleic acid assembly. The embodiment depicted in FIG. 4B further resembles that of FIG. 3B, with fluorophores 205 and 207 attached to the tubulin subunits 203.

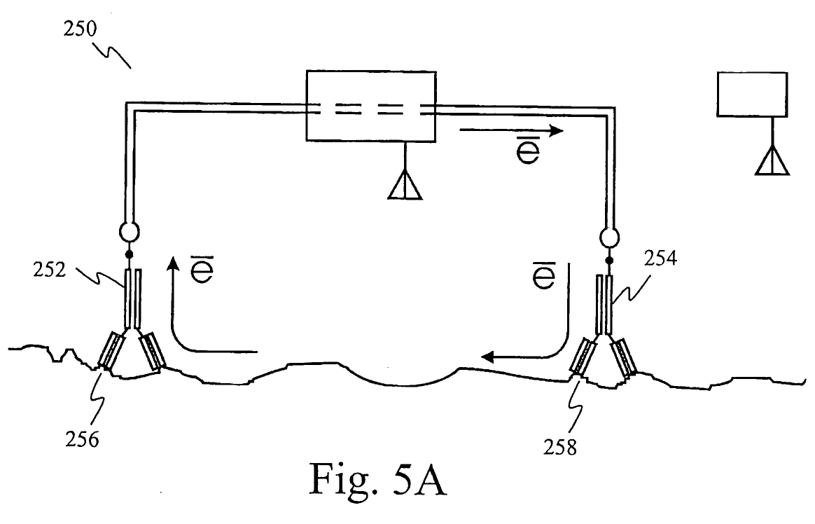

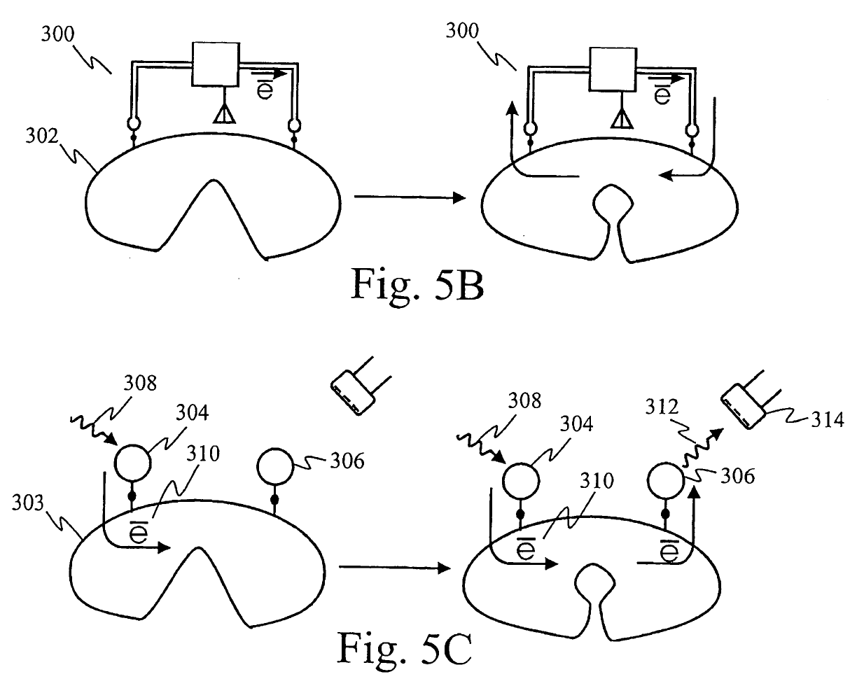

In another embodiment, depicted in FIG. 5A, both ends of the electrode 250 are attached to antibodies or antibody fragments 252 and 254 which recognize a given antigen or antigens 256 and 258. The transceiver would be able to measure changes in electrical properties of the element to which both antibody/antibody fragments are attached.

In yet another embodiment, depicted in FIG. 5B, both ends of the transceiver 300 are attached to a single protein 302, such as an enzyme or transcription factor. Such proteins will change their physical conformation depending on whether they are active or at rest, which will in turn change their conductivity. Such changes could be detected by the attached transceiver 300. In still another embodiment, a transceiver is attached to a protein such that constant electrical current flows through the protein, e.g., a transcription factor.

FIG. 5C is a schematic diagram depicting yet another embodiment, similar to FIG. 4B, wherein fluorophores 304 and 306 are attached to a single protein 303 which may change its physical conformation depending on its activity. When the protein 303 is in one state of activity, excitatory energy 308 may be used to excite fluorophore 304 and release an electron 310. Electron 310 will not be able to reach fluorophore 306 due to the lower conductivity of the protein. However, upon a change of the physical state of the protein, e.g. when an enzyme binds a substrate, excitement of fluorophore 304 will lead to conduction of electron 310 through the protein and subsequent excitement of fluorophore 306. Wavelength emissions 312 from fluorophore 306 can then be detected with detector 314.

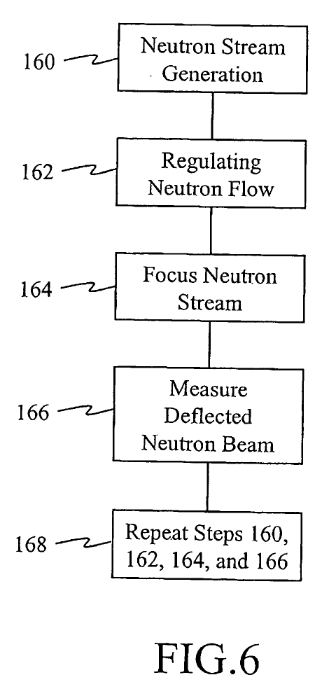

FIG. 6 is a flow diagram of a process for imaging chromosomal events in biological systems. In the first step of the process depicted in FIG. 6, in step 160, a neutron stream is produced by conventional means such as, e.g., a cyclotron. See, e.g., U.S. Pat. Nos. 5,699,394, 5,386,114, 4,853,550, 4,701,792, 4,176,093, the entire disclosure of each of these United States patents is hereby incorporated by reference into this specification.

Thereafter in step 162 of the process, the flow of the neutron stream is regulated by means of, e.g., supercooled fluid. See, e.g., U.S. Pat. Nos. 5,872,826, 5,610,956, 5,367,547, 5,174,945, 5,128,097. The entire disclosure of each of these United States patents is hereby incorporated by reference into this specification.

In step 164 of the process, the slowed neutron stream is focused onto a nucleus of a cell which one wishes to scan. See, e.g., U.S. Pat. Nos. 6,054,708, 5,658,273, 5,076,993, etc., the entire disclosure of each of which is hereby incorporated by reference in to this specification. In this step 164, the cell nucleus is bombarded with the slowed neutron stream, which is preferably focused onto desired target chromosomes.

In one embodiment, streams of neutrons are simultaneously focused on different portions of the nucleus to produce a three-dimensional image. In this embodiment, one may scan the beams over the portions of the nuclei being examined to produce a real time image.

The neutrons impacting the cell nuclei are transmitted and deflected. In step 166, these deflected neutron beams are measured and analyzed. The extent of the transmission and deflection of the neutron beam, and the intensity of each, varies with the mass contacted by the neutron beam. Thus, one can determine deflection patterns of neutron beams for standardized DNA samples and compare this data with the deflection patterns produced with any particular DNA sample. This data correlation will indicate whether any particular DNA is aberrant, and/or is undergoing an event such as replication or transcription.

The transmitted and/or deflected neutron beams are analyzed in a controller comprised of a neutron detector, a monitor, image processing software, so that the shape, structure, motion, configuration, and relative positioning of the chromosomal moieties within the nuclei may be viewed and evaluated. By way of illustration and not limitation, one may thus examine, evaluate, and quantify phenomena such as the positioning of the histones and nucleosomes within chromatin, the spatial relationship between chromosomes, looping of the DNA, pseudo knot formation within the DNA, supercoiling within the DNA, unwinding of the DNA, the energy state of the DNA, and any other structural property normally or abnormally exhibited by chromatin. The observation, evaluation, and compilation of these properties allow one to prepare a database of normal and aberrant states of the cell.

In step 168, steps 160, 162, 164, and 166 are repeated as the focus of the neutron beam is changed. Thus, one can scan a DNA segment in real time and instantaneously generate data to indicate the condition, and what events, if any, such DNA segment is undergoing.

The steps 160 through 168 can be repeated with a wide variety of standardized DNA samples, and test DNA samples, to produce a database that can thereafter be correlated with any particular set of readings. By way of illustration, such a database will have data regarding the phenotypes of individuals with specific maladies, such as disease state, age, etc. When a scanning of any particular DNA sample matches such a malady phenotype, one then gains an indicium of the possibility of such malady existing or developing.

In practicing the techniques described in this specification, one may use procedures described in prior art patents. Thus, one should refer to U.S. Pat. No. 5,612,468 (Pteridine nucleotide analogs as fluorescent DNA probes); U.S. Pat. No. 5,846,708 (Optical and electrical methods and apparatus for molecule detection); U.S. Pat. Nos. 4,447,546, 4,582,809, 4,909,990, 5,776,672 (Gene detection method); U.S. Pat. No. 6,146,593 (High density array fabrication and readout method for fiber optic biosensor); and U.S. Pat. No. 6,146,593. The entire disclosure of each of these United States patents is hereby incorporated by reference into this specification.

As indicated elsewhere in this specification, the transceivers 74 (see FIG. 3A) are preferably nanotubes. As used this specification, the term nanotube includes a nanoparticle, which can be fabricated from gold, from carbon, from other materials, and may be fabricated in substantially any shape.

In one embodiment, the nanoparticle consists essentially of gold. In another embodiment, the nanoparticle is a nanotube which can contain a single wall, or a double wall, or a multiplicity of walls. These walled nanotubes are well known to those skilled in the art. See, e.g., U.S. Pat. Nos. 6,187,760, 6,187,823, 6,183,174, and 6,159,742. The entire disclosure of each of these United States patents is hereby incorporated by reference into this specification.

The invention has been described by reference to deoxyribose nucleic acid structures, as well as ribonucleic acid structures. It is equally applicable to protein structures, to which an antibody may be bonded in conventional manner to different moieties on the protein entity.

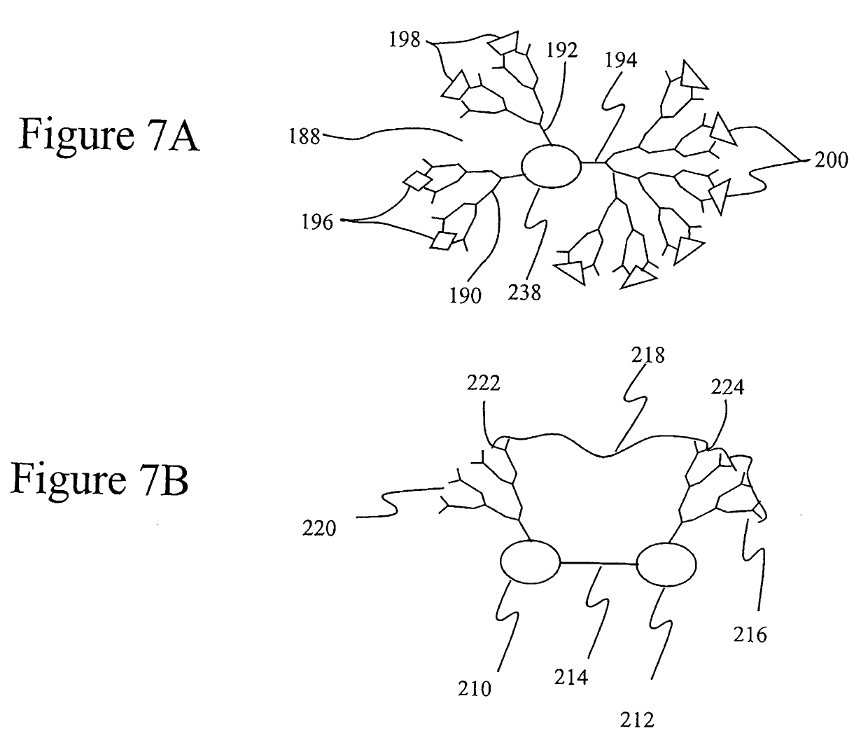

As another embodiment of this invention one can reference FIG. 7 the polymeric molecules defined as dendrimers can be used as a transceiver. The term dendrimers as defined within can be made of any number of polymers or metals. These may include but are not limited to in this invention, polyamidoamine (PAMAM), polypropylamine (POPAM), polyethylenimine, poly(propylene imine), iptycene, aliphatic poly(ether), and/or aromatic polyether dendrimers. For metal formation of dendrimers one can reference U.S. Pat. No. 6,618,941, “Method of forming freestanding metal dendrites”. The entire disclosure of this United States patents is hereby incorporated by reference into this specification.

The term dendrimers as defined within can be of any shape. These geometrical variations include but are not limited to in this invention, star shaped, rod shaped, starburst polymers, dense star polymers and bridge dense star polymers. Description of the fabrication and characterization of dendrimers are extensively described in the patent literature (i.e. see U.S. Pat. No. 5,560,929, “Structured copolymers and their use as absorbents, gels and carriers of metal ions” or U.S. Pat. No. 4,946,824, “Connected branch copolymers, methods for their production, and copying materials including same”). The entire disclosure of each of these United States patents is hereby incorporated by reference into this specification.

Dendrimers can be conjugated to any number of planar surfaces for use in an array of molecular detection devices. Conductive elements, transceivers, quantum dots and metallic particles (to also include nanoparticles) can all be embedded within dendrimer layers in solution as is described for metal ions in U.S. Pat. No. 5,560,929, “Structured copolymers and their use as absorbents, gels and carriers of metal ions”, the disclosure of which is incorporated herein by reference.

FIG. 7A depicts a nucleic acid-dendrimer controller complex with different affinities for specific and separate nucleic acid sequences. In one embodiment, conductive dendrimers may be functionalized with the appropriate groups to bind a number of nucleic acid sequences. In FIG. 7A, three separate dendrimers 190, 192, and 194 are shown to bind three separate nucleic acid constructs 196, 198, and 200. The nucleic acid sequences can be specific to one sequence in the genome, an entire gene, the cDNA of any organism or any genetic elements from different species and a mixture thereof. The nucleic acid sequences can be in the form of single stranded deoxyribonucleic acid (ssDNA), single stranded ribonucleic acid (ssRNA), double stranded deoxyribonucleic or ribonucleic acid (dsDNA, dsRNA).

The fabrication and use of conductive polymer dendrimers has also been previously described (see for example U.S. Pat. No. 6,043,336, “Electrically conducting dendrimers”). In one embodiment, dendrimers may also be used in part to form circuits in a similar fashion as shown in FIGS. 3A and 4A. In this embodiment, the dendrimers 190, 192, and 194 will be conductive dendrimers, which are linked in the center via different functional groups than are used on the opposing branches of the dendrimers 190, 192, and 194. The three dendrimers are attached to a nanosensor-microprocessor 238 via functionalized moieties conjugated to the dendrimers during preparation of nucleic acid-dendrimer controller complex 188. The link 238 between the conductive polymer dendrimers can also be a fluorophore or quantum dot embedded within a polymer bridge.

The emission of energy from an external source (not shown) or the controller 238 will be absorbed by the three separate nucleic acid sequences 196, 198, and 200. Energy in the form of electrons will travel to the controller via the conductive dendrimers 190, 192, and 194. The magnitude of the signals acquired from the three separate dendrimers will be integrated by the link 238. Integration of signals by link 238 can be performed by a controller, nanosensor, fluorophore, chromaphore, or quantum dot. This integration process can be performed with any plurality of separate nucleic acid sequences and dendrimers. The resultant output of the controller will provide a network analysis characteristic of the specified nucleic acid sequences.

FIG. 7B depicts a multi-dendrimer-nucleic acid controller complex with different affinities for specific nucleic acid sequences on the same strand. Referring to FIG. 7B, and in one embodiment depicted therein, the entire cDNA of 218 is attached to two separate dendrimers 216 and 220. The attachment points 222 and 224 are site specific sequences within the cDNA 218. In this embodiment, the dendrimers 216 and 220 will be conductive dendrimers, which are linked to their respective links 210 and 212 via different functional groups than are used on the opposing branches. The links 210 and 212 can be a controller, nanosensor, fluorophore, chromaphore, or quantum dot. The links 210 and 212 can be connected via an electrically conductive element or optical path 214.

In one embodiment, as depicted in FIG. 7B, the emission of energy from an external source (not shown) or the links 210 and 212 will be absorbed by the cDNA 218. Energy in the form of electrons will travel to the links 210 and 212 via the conductive dendrimers 220 and 216. The magnitude of the signals acquired from the two separate dendrimers will be integrated by the links 210 and 212. This integration process in this embodiment can be performed with any double stranded or single stranded nucleic acid sequence. The resultant output of the controller will provide a characteristic analysis of the interaction of specified sites within a nucleic acid sequence.

In another embodiment, a dendrimer is embedded within two separate regions of a polypeptide matrix to form a dendrimer-polypeptide complex. The polypeptide matrix component of the complex may be comprised of any protein known to form matrices or networks. Attachment of dendrimers to polypeptides has been performed and is known to those skilled in the art. In U.S. Pat. No. 6,083,708 (“Polypeptide: dendrimer complexes”) the inventors describe complexes capable of binding of dendrimers to selective regions of polypeptides with varying affinity. The entire disclosure of this patent is incorporated herein by reference.

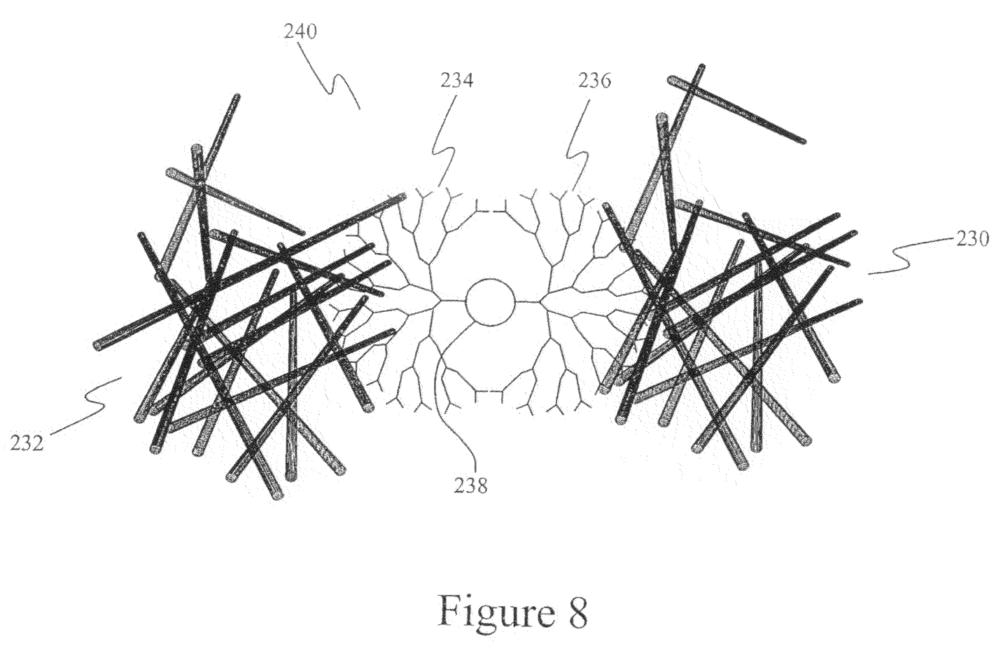

In one embodiment of the instant invention, the polypeptide matrix is an array of collagen, which is in the form of a gel. Other matrices may include fibronectin, vitrogen, laminin or any other possible extracellular matrix component. The polypeptide matrix or array may be found in vivo or in vitro. In one embodiment and as depicted in FIG. 8, the tubular structure forming the regions 230 and 232 are microtubule or actin networks of a portion of the cytoskeleton. These regions 230 and 232 are linked by two half-star conductive polymer dendrimers 234 and 236. These half-star conductive polymer dendrimers 234 and 236 are attached in the center to link 238.

In this embodiment, the dendrimers 234 and 236 will be conductive dendrimers, which are attached to link 238 via different functional groups than are used on the opposing branches of dendrimers 236 and 238. The two half-star dendrimers 234 and 236 are attached to the link 238 via functionalized moieties conjugated to the dendrimers during preparation. The link 238 between the conductive polymer dendrimers 234 and 236 can also be a fluorophore, nanosensor, chromophore, or quantum dot embedded within a polymer bridge. The dendrimer-matrix-link complex 240, where link 238 can be a chromophore, fluorophore, or quantum dot.

It is to be understood that any of these embodiments described within this specification can be used in vitro or in vivo. For the in vivo case, dendrimer-link complexes may be formed as an emulsification and then encapsulated into lipid constructs capable of insertion within a cell. Techniques such as these are described earlier in this specification. Dendrimer-matrix-link complexes can be used in a similar fashion in arrays for diagnostics and drug-discovery as described in U.S. Pat. No. 6,471,968 (“Multifunctional nanodevice platform”), the disclosure of which is incorporated herein by reference.

The cytoplasmic network of microtubules or actin may vary in different cell types. These variations may be in protein concentration, alignment and directionality of the network, or the binding efficiency of subunits of the network. The defined variations will result in different signal signatures detected by the link 238. Therefore, this technique may provide a new modality for distinction between different cell types and their micro-environments in normal physiological processes and in disease.

It is to be understood that the aforementioned description is illustrative only and that changes can be made in the apparatus, in the ingredients and their proportions, and in the sequence of combinations and process steps, as well as in other aspects of the invention discussed herein, without departing from the scope of the invention as defined in the following claims.

1-63. (canceled)

64. A system for determining a physical property of a nucleic acid polymer or an amino acid polymer, comprising:
a polymer selected from the group consisting of a nucleic acid polymer and an amino acid polymer;
a first transceiver for receiving a first signal from an energy source and introducing said first signal to the polymer, wherein the first transceiver is conjugated to the polymer;
a second transceiver for receiving a second signal from the polymer, wherein the second transceiver is conjugated to the polymer;
a device for producing a third signal containing information regarding a difference between the first and second signals.

65. The system according to claim 64, wherein the signal provided by the energy source is selected from the group consisting of mechanical, ultrasonic, light, laser, magnetic and radio wave energy.

66. The system according to claim 64, wherein each of the first and second transceivers is independently selected from a nanotube, a nanoparticle, a fluorophore or a dendrimer.

67. The system according to claim 64, wherein the polymer is an amino acid polymer, and the amino acid polymer is a protein or a peptide.

68. The system according to claim 64, wherein at least one of the first and second transceivers comprises gold.

69. The system according to claim 64, wherein at least one of the first and second transceivers is a carbon nanotube.

70. The system according to claim 64, wherein the polymer is a nucleic acid polymer which is a single-strand polymer or a double-strand polymer.

71. The system according to claim 64, wherein the first and second transceivers and the signal-producing device are transfected into a cell.

72. The system according to claim 71, wherein said cell is located in a cell culture apparatus.

73. The system according to claim 64, wherein the assembly is located within an organism comprised of one or more cells.

74. The system according to claim 64, wherein the polymer is an amino acid polymer comprised of a proximal end and a distal end, and wherein a first antibody is bound to a first antigenic site of the amino acid polymer, and a second antibody is bound to a second antigenic site on the amino acid polymer.

75. The system according to claim 64, wherein the produced signal is a radio frequency signal.

76. The system according to claim 64, further comprising a device for receiving the third signal and determining a physical property of the nucleic acid polymer or amino acid polymer.

77. The system according to claim 64, wherein at least one of the first and second transceivers is a dendrimer consisting essentially of a conductive polymer.

78. The system according to claim 77, wherein the dendrimer has a shape selected from the group consisting of a rod shape form, a spiral shape form, a star shape form, a starburst shape form and a spherical shape form.

79. The system according to claim 64, wherein the energy detection device is selected from the group consisting of a microvoltage potential detector, a microcurrent detector, an ultrasonic transducer, a microprocessor control unit, a nanoparticle sensor, a fluorophore, a chromophore, a nanotube or a quantum dot.
